(19) United States
(12) Patent Application Publication
(10) Pub. No.: US 2007/0215155 A1
(43) Pub. Date: Sep. 20, 2007

(54) Ventilator with memory for operating data

(76) Inventors: Thomas Marx, Hamburg (DE); Jacqueline Stuhlmacher, Kiel (DE); Johannes Jensen, Kiel (DE)

Correspondence Address: Friedrich Kueffner, Suite 910, 317 Madison Avenue, New York, NY, 10017, US

(21) Appl. No.: 11/489,250

(22) Filed: Jul. 19, 2006

(30) Foreign Application Priority Data
Mar. 16, 2006 (DE) 10 2006 012 727.7
Jul. 19, 2006 (DE) 10 2005 034 238.8

(51) Int. Cl. A61M 16/00; A62B 7/00

(52) U.S. Cl. 128/204.21; 128/204.18

(57) ABSTRACT

A ventilator with memory for operating data has a control unit for a breathing gas source. The operating data memory is provided to store the current changeable operating data. The control unit is provided with a data-saving function which stores the current operating parameters during an intermission in the operation of the device. The control unit also has a memory readout function, by which the stored operating data can be read out after the end of an intermission to allow the ventilator to resume operation in correspondence with the saved operating data.

### BACKGROUND OF THE INVENTION

1. Field of the Invention

The invention relates to a ventilator with memory for operating data and with a control unit for a source of breathing gas.

2. Description of the Related Art

Ventilators of this type are used, for example, for the artificial ventilation of patients and are typically equipped with a breathing gas source such as a fan. The breathing gas is typically supplied to the user through a breathing gas hose, which is connected on the patient side to a breathing mask or some other type of patient interface.

While being ventilated, the user cannot usually converse, drink, or eat, because of the presence of the patient interface and/or the pressure of the breathing gas. Because the user must open his mouth to speak, the breathing gas, which is under pressure, would escape noisily through his mouth unless the ventilator is turned off briefly and/or the breathing mask and/or the patient interface is removed. It is therefore frequently impossible for the user to conduct a normal conversation. The process of turning off the ventilator and removing the patient interface is time-consuming, however, and is frequently perceived by the user as inconvenient and annoying.

Another problem is that, after the occurrence of some type of problem such as failure of the power supply, operating parameters individually selected by the patient are usually no longer available, and the unit must be reset to a default state defined by the manufacturer or medical personnel. The user must in this case re-enter all of the individually selected settings for the operating parameters.

### SUMMARY OF THE INVENTION

The object of the present invention is to provide a ventilator of the type indicated above in such a way that it can be used with greater convenience.

This object is met according to the invention in that the operating data memory is designed to store the current changeable operating data, and in that the control unit is provided with a data-saving function, which stores the current operating parameters during an intermission in the operation of the device, and with a memory readout function, by which the stored operating data can be read out after the end of the intermission to allow the ventilator to resume operation in correspondence with the saved operating data.

Because the current operating data are saved to memory, it is possible for the user to interrupt briefly the supply of air through the ventilator so that he can speak, drink, eat, exchange kisses, etc., without the need to turn off the ventilator and/or without the need to remove the patient interface.

An advantageous embodiment of the invention is realized in that a device, by means of which the user can switch the ventilator to “pause” mode for a period of time definable by the user, is provided in the area of the unit in the form of a remote control device or a wristband, possibly with a clip, or in the form of an automatic function in the ventilator. Pause mode can be activated, for example, acoustically, mechanically, pneumatically, optically, or electronically.

The user can activate the function by, for example pressing on an actuating surface or by giving an acoustic signal.

Pause mode is characterized in that the unit is still functioning, but the air supply to the patient is at least reduced.

Alternatively, the air supply to the user is reduced to zero in pause mode.

If desired, it is also possible to provide the user with the ability to define his own pause mode. For example, a predetermined residual pressure can be maintained during pause mode.

It is provided that the user can set the duration of pause mode by making use of a selection function. In terms of the control technology, a time counting function then starts running in the unit, the duration of which corresponds essentially to the value entered by the user. After the time counting function has completed its run, the unit switches back to operating mode and runs under the same conditions as those present before pause mode was activated.

According to another embodiment, the user can initiate pause mode by making use of a selection function. Pause mode then remains activated until the patient terminates pause mode by making use of a selection function. In terms of the control technology, the unit then stores the operating settings under which it was running before pause mode was initiated, and then, after pause mode has been terminated, returns immediately to the active operating mode.

According to another embodiment, the user can initiate pause mode by making use of a selection function. Pause mode then remains activated until the patient terminates pause mode by making use of a selection function. In pause mode, the unit remains in the operating mode which was present before the initiation of pause mode. The breathing gas is merely bypassed around the patient. Upon termination of pause mode, the unit returns immediately to the active operating mode.

In a preferred embodiment, the unit initiates pause mode by itself, i.e., automatically, by means of a selection circuit when events are recognized which call for pause mode. Pause mode then remains activated until either the user terminates pause mode by making use of a selection function or until the unit no longer registers the event causing pause mode to be activated.

Upon termination of pause mode, the unit returns immediately to the active operating mode.

The control unit for automatic pause mode contains an analyzer for detecting at least one event, and the analyzer is connected to the control unit in such a way that, when an event is recognized, pause mode is activated.

It is provided in particular that, when an event is recognized, a sequence control circuit provided as part of the control technology activates pause mode.

In the inventive device and in the inventive method, advantage is taken of the fact that specific events lead to typical effects on the ventilation parameters, which can be measured by suitable measuring devices. The typical changes over time which occur in the ventilation parameters can be correlated with specific events. This make it possible for the changes which occur in a signal over time to be evaluated automatically and thus for the control system to identify the event in question.

Corresponding specific events which can be recognized on the basis of the changes in the signal include, for example, mouth expiration, mouth breathing, leakage, swallowing, speaking, sneezing, and coughing. When events of this type are detected automatically, it is possible to modify the pressure control in such a way that the measurement parameters, which are evaluated by the control unit under normal conditions as a basis for either raising or lowering the pressure but which can no longer be evaluated reliably during the occurrence of one of the events in question, are used by the control system for the duration of the occurrence of the event to activate pause mode.

The method will be carried out typically in that the control system is designed to implement CPAP, APAP, bi-level, home, hospital, intensive-care, and/or emergency type ventilation.

According to an embodiment, the analyzer is designed to evaluate a flow curve.

It is also possible for the analyzer to be designed to evaluate a pressure curve.

A variant of the method consists in that the analyzer is designed to evaluate inspiration phases.

It is also possible to design the analyzer to evaluate expiration phases.

According to a simple principle upon which evaluation can be based, the analyzer is designed to evaluate amplitude values.

It is also possible to design the analyzer to evaluate output values of the energy uptake of the breathing gas source.

According to another design variant, the analyzer has a reference value comparator.

A frequency-dependent evaluation of the signal is supported by subjecting the measured pressure signal to bandpass filtering.

In particular, it is possible to define a frequency band for the bandpass filtering in such a way that the amplitude of a volume vibration generated by the unit is measured. An excitation signal suitable for bandpass filtering can be provided by a membrane pump, which generates the volume vibration.

A good compromise between realization with simple equipment on the one hand and high-quality evaluation of the excitation signal on the other consists in generating the volume vibration at a frequency of approximately 20 Hz.

According to a typical evaluation method, an expiratory narrowing of the airways is recognized on the basis of an expiratory increase in the amplitude of the pressure vibration in comparison with a reference value.

According to a special variant of event detection, individual or cumulative expiratory narrowings are evaluated as events.

If events are stored and evaluated, it is possible, by means of a self-learning system, to refine the quality of the reaction of the ventilator and of the recognition of events.

The ventilator has a pressurized gas source which can be connected to a patient interface, a control unit for the pressurized gas source, and a measuring device for detecting at least one breathing parameter. The control unit is provided with an actuating device for changing the pressure provided by the pressurized gas source as a function of the evaluation of the measured breathing parameter. The control unit has an analyzer for detecting at least one event. The analyzer is connected to the control unit in such a way that, when an event is detected, the ventilator is switched to pause mode.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a perspective view of a ventilator with breathing gas hose and breathing mask;

FIG. 2 shows a modified form of the ventilator according to FIG. 1 with a pause button;

FIG. 3 shows the ventilator according to FIG. 2 with a humidifier attached;

FIG. 4 shows the ventilator according to FIG. 2 with an oxygen feed valve attached;

FIG. 5 shows the ventilator according to FIG. 4 in a perspective view;

FIG. 6 is a perspective view of a ventilator installed in a carry-case with indication of various details;

FIG. 7 is a perspective view of a transport device for a mobile ventilator;

FIG. 8 shows a transport device according to FIG. 7 after the installation of the ventilator according to FIG. 6;

FIG. 9 shows a pause button which can be mounted on a wristband;

FIG. 10 shows the pause button according to FIG. 9 with a different set of dimensions;

FIG. 11 shows the pause button according to FIG. 10 after it has been attached to the wrist of a patient; and

FIG. 12 shows a patient with a clip-like pause button.

### DETAILED DESCRIPTION OF THE INVENTION

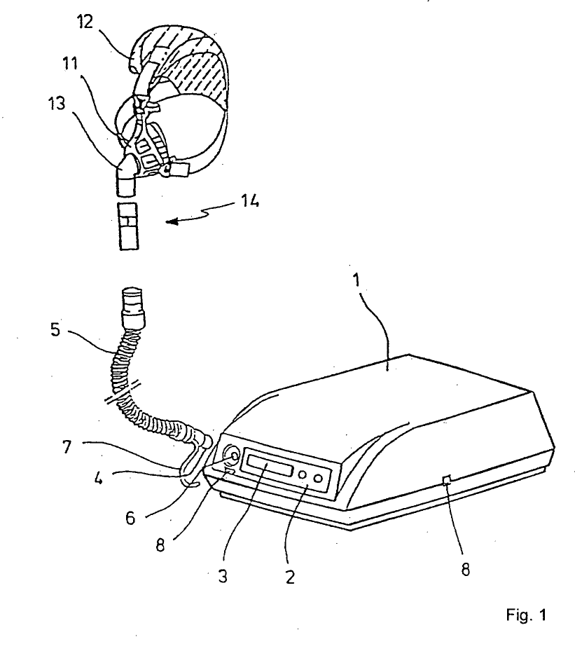

FIG. 1 shows the basic structure of a ventilator. A breathing gas pump is installed in the interior of the equipment housing (1), which has a control panel (2) and display unit (3). A connecting hose (5) is connected by means of a coupling (4). An additional pressure-measuring hose (6), which can be connected to the equipment housing (1) by means of a pressure feed connector (7), can extend alongside the connecting hose (5). The equipment housing (1) has an interface (8) for the transmission of data. An expiration element (9) is provided in the area of the connecting hose (5) facing away from the equipment housing (1). FIG. 1 also shows a patient interface (10), which is designed as a nose mask. The mask can be held in place on the patient's head by a hood (11). In the area facing the connecting hose (5), the breathing mask (10) has a connector piece (12).

In the area of the equipment housing (1), a pause button (13) is provided, so that the ventilator can be switched manually to pause mode. According to another embodiment, the pause button (13) can be located in the area of the control panel (2) or be designed as an external control element.

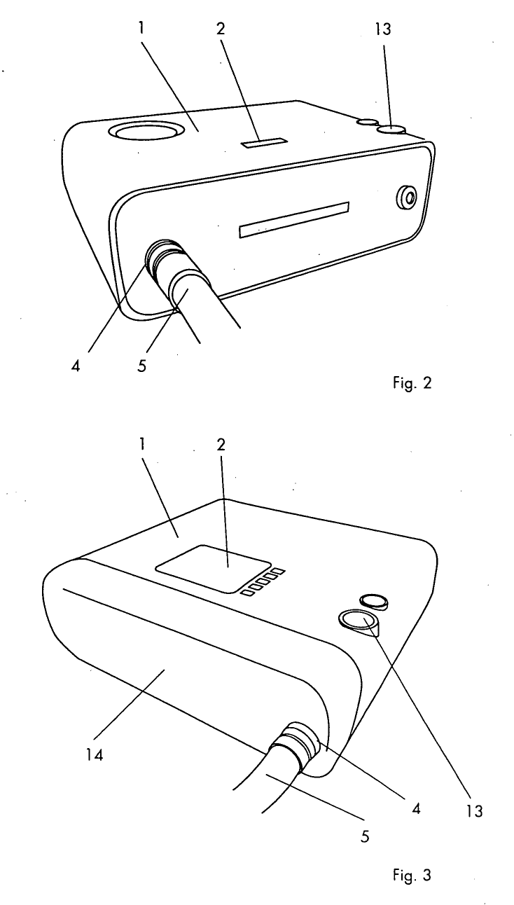

FIG. 2 shows a different embodiment of the ventilator. The pause button (13) here is positioned in an upper side area of the equipment housing (1).

To prevent the airways from drying out, it is advisable, especially during prolonged periods of artificial ventilation, to humidify the breathing air according to FIG. 3. Adding moisture to the breathing air in this was can also be realized in other applications. To provide the necessary moisture, adaptable breathing air humidifiers (14) are usually installed in the airway between the ventilator and the patient.

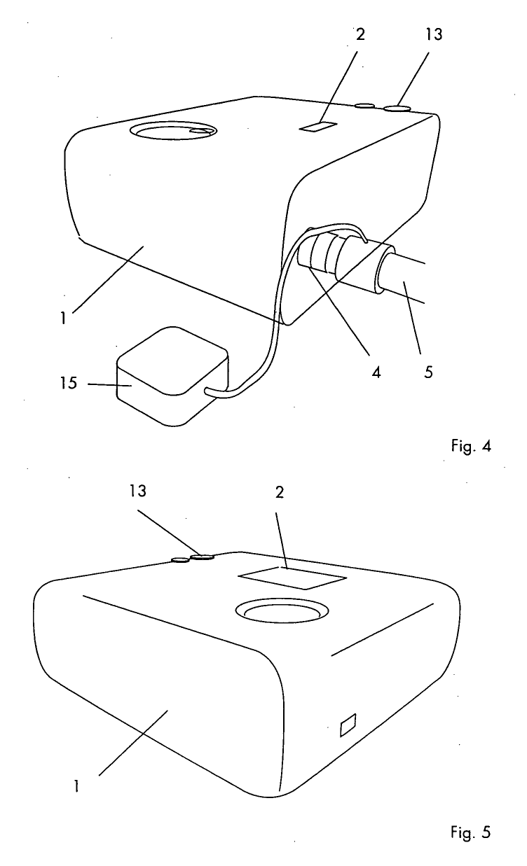

In addition, it is also possible according to FIG. 4 to adapt an oxygen feed valve (15) to increase the amount of oxygen in the breathing gas supplied to the user.

FIG. 5 shows a perspective view, from the rear, of the arrangement according to FIG. 4. It is possible to see here in particular the contours of the equipment housing (1) with particular clarity.

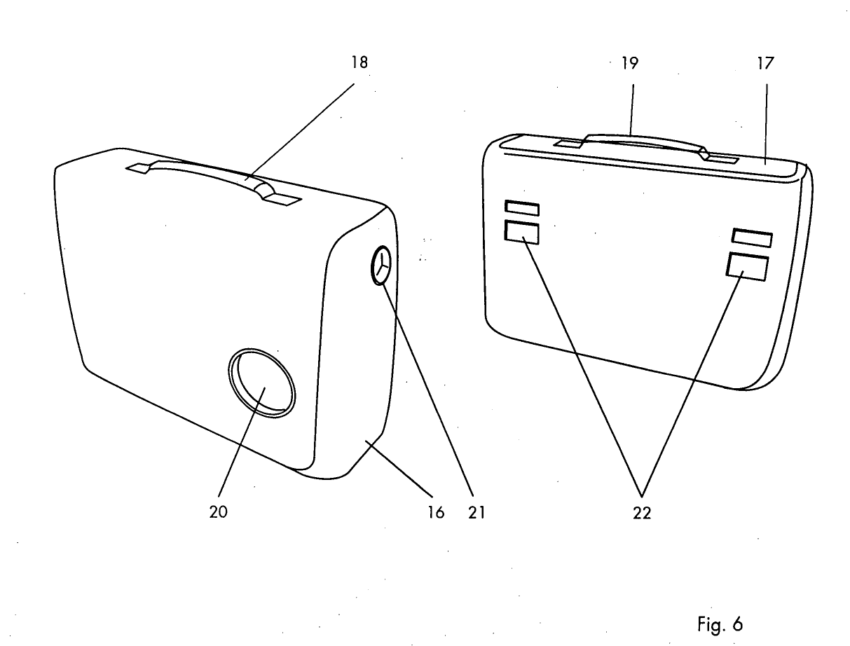

FIG. 6 shows a ventilator, the functional components of which are installed in two different cases (16, 17). Packing the ventilator in cases (16, 17) facilitates mobile applications. The cases (16, 17) are equipped with handles (18, 19). In addition, an air inlet (20) and a hose connection (21) can also be seen. The cases (16, 17) can be held in place by the use of pocket connectors (22).

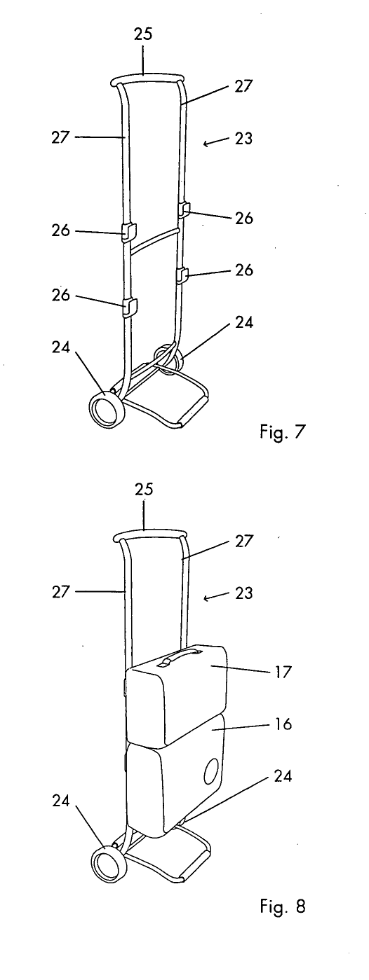

FIG. 7 shows a transport device (23) for the cases (16, 17). The transport device (23) is built in a manner similar to a bag truck with wheels (24) and a handle (25). Retaining elements (26) are provided in the area of the vertical struts to position the cases (16, 17).

FIG. 8 shows the transport device (23) after the cases (16, 17) have been fastened in place. The cases (16, 17) are held in place by engaging the retaining elements (26) in the pocket connectors (22).

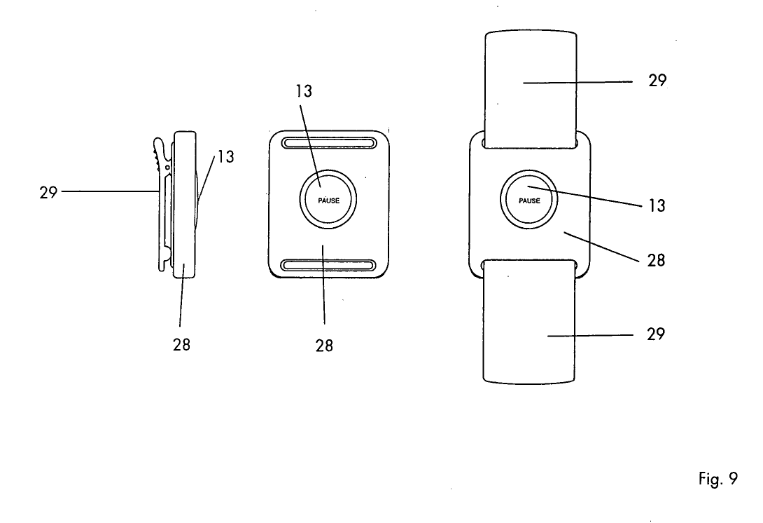

FIG. 9 shows an embodiment in which the pause button (13) is mounted in a carrier (28), to which a wristband (29) is attached. The wristband (29) can, for example, be wrapped around the user's wrist and makes it possible for the pause function to be initiated remotely.

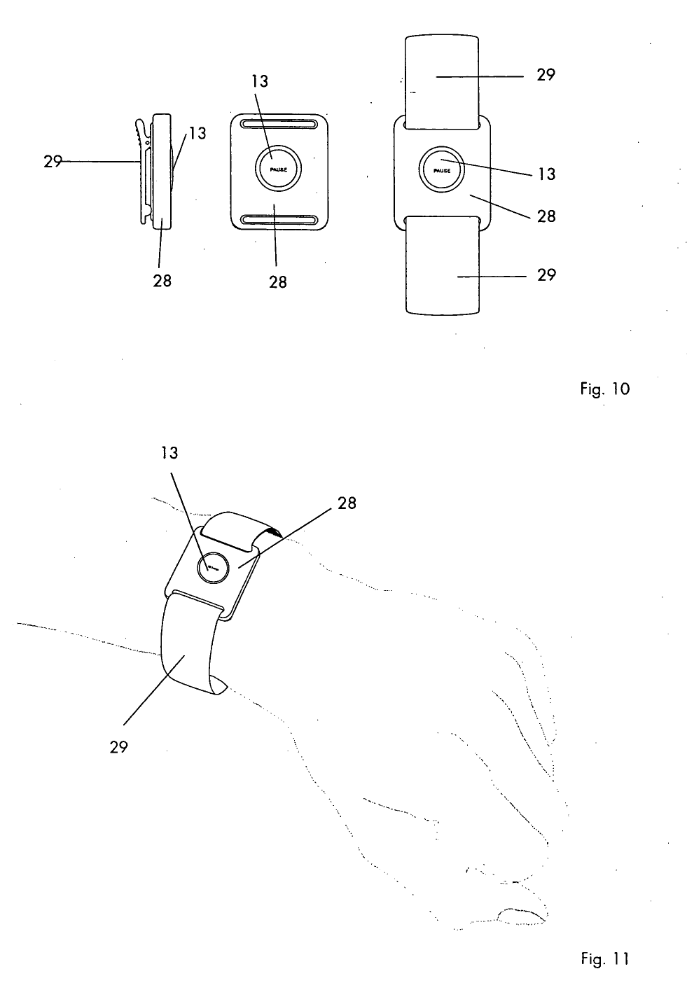

FIG. 10 shows a carrier (28) with pause button (13) similar to FIG. 9 but with different dimensions.

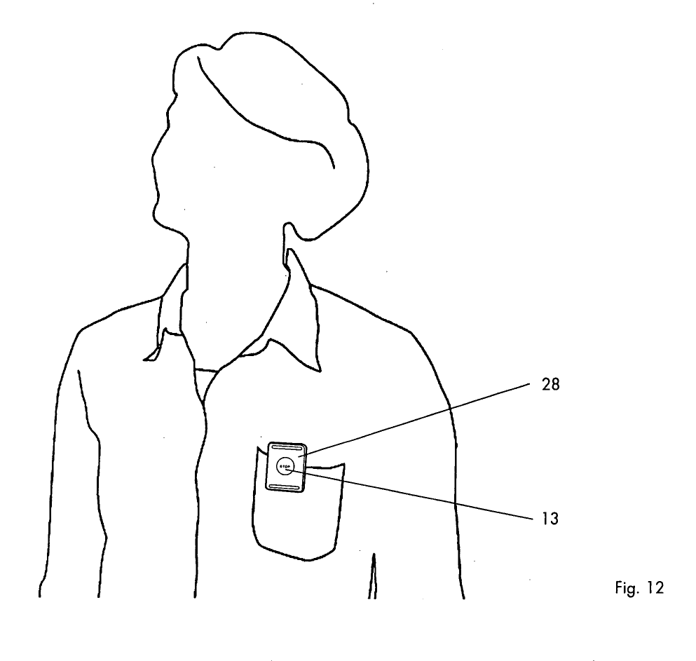

FIG. 11 shows how the carrier (28) with pause button (13) would look after the wristband (29) has been placed around the user's wrist.

FIG. 12 shows an embodiment in which the carrier (28) with pause button (13) is designed as a clip. It is therefore possible to attach the pause button to the pocket of a shirt or jacket, for example.

While specific embodiments of the invention have been shown and described in detail to illustrate the inventive principles, it will be understood that the invention may be embodied otherwise without departing from such principles.

1. A ventilator with memory for operating data, the ventilator comprising a control unit for a breathing gas source, wherein the operating data memory is adapted to store the current changeable operating data, and the control unit is provided with a data-saving function, which stores the current operating parameters during an intermission in the operation of the device, and a memory read-out function, for reading out the stored operating data after the end of the intermission such that the ventilator resumes operation in correspondence with any saved operating data.

2. The ventilator according to claim 1, comprising means for activating the data-saving function an operating problem occurs.

3. The ventilator according to claim 1, characterized in that the data-saving function is activated when a pause function is actuated manually.

4. The ventilator according to claim 1, wherein at least one pause mode is provided.

5. The ventilator according to claim 1, wherein a pause mode can be preselected by the user.

6. The ventilator according to claim 1, wherein a pause mode can be initiated automatically by the unit upon recognition of predefined events.

7. The ventilator according to claim 1, wherein, during pause mode, the user can speak and/or sneeze and/or drink and/or eat and/or exchange kisses and/or cough and/or telephone and/or read and/or take medications with essentially no interference from the ventilator.

8. The ventilator according to claim 1, wherein, during pause mode, the ventilator provides the user with a breathing gas pressure which is lower than the original pressure.

9. The ventilator according to claim 1, wherein, during pause mode, the ventilator provides the user with a breathing gas pressure of less than 6 mbars.

10. The ventilator according to claim 1, wherein, during pause mode, the ventilator provides the user with a breathing gas pressure of less than 4 mbars.

11. The ventilator according to claim 1, wherein, during pause mode, the ventilator provides the user with a breathing gas pressure of less than 2 mbars.

12. The ventilator according to claim 1, wherein, during pause mode, the ventilator provides the user with a breathing gas pressure of essentially 0 mbar.

13. The ventilator according to claim 1, wherein the pause function can be initiated by remote control.

14. The ventilator according to claim 1, comprising the pause function can be initiated by a pause button (13) located in the area of a wristband (29) for initiating the pause function.

15. The ventilator according to claim 1, comprising the pause function can be initiated by a pause button (13) located in the area of a clip for initiating the pause function.

16. The ventilator according to claim 1, wherein the pause function can be activated for a preselected period of time.

17. The ventilator according to claim 1, comprising an analyzer is provided for the automatic detection of the termination of a pause state.

18. The ventilator according to claim 1, wherein a microphone is connected to the analyzer for the detection of events.

19. The ventilator according to claim 1, wherein the analyzer is connected to a memory in which sound patterns are stored.

20. The ventilator according to claim 1, wherein the pause function can be deactivated for a preselected period of time.

21. A method for controlling a ventilator, comprising preselecting a pause mode by making use of at least one selection means.

22. A method for controlling a ventilator, comprising hardware for automatically activating pause mode in response to events by means of at least one selection circuit.

23. A method according to claim 21, comprising activating pause mode automatically on the basis of the detection of an event.

24. A method according to claim 21, wherein a user activates the pause mode.

25. A method according to claim 21, wherein a user activates the pause mode by actuating a means provided in the area of the ventilator.

26. A method according to claim 21, wherein a user activates the pause mode by actuating a means connected to the ventilator by a cable.

27. A method according to claim 21, wherein a user activates the pause mode by actuating a means which communicates with the ventilator in wireless fashion.

28. A method according to claim 21, wherein at least one event is detected by a microphone.

29. A method according to claim 21, wherein a detected sound is compared with a stored sound pattern.

30. A method according to claim 21, wherein the pause mode can be deactivated under preselected conditions.

31. Means which communicates wirelessly with a ventilator, wherein a user activates the pause mode of the ventilator by making contact with the means.
